(19) United States
(12) Patent Application Publication
(10) Pub. No.: US 2007/0280866 A1
(43) Pub. Date: Dec. 6, 2007

(54) Cleaving Of Oligomeric (Meth) Acrylic Acid In A Liquid Phase Under Pressure

(76) Inventors: Torsten Balduf, Pfungstadt (DE); Jurgen Mosler, Castrop- Rauxel (DE); Thomas Rathke, Mandeville, LA (US); Dennis Thong Yu-Chiang, Marl (DE); Axel Kobus, Bochum (DE); Rudolf Burghardt, Recklinghausen (DE); Michael Bernd Fricke, Gescher (DE); Stefan Nordhoff, Recklinghausen (DE)

Correspondence Address: SMITH MOORE LLP, P.O. BOX 21927, GREENSBORO, NC, 27420, US

(21) Appl. No.: 10/589,775

(22) PCT Filed: Feb. 18, 2005
(86) PCT No.: PCT/EP05/01705

§ 371 Date: Jun. 15, 2007

(30) Foreign Application Priority Data
Feb. 19, 2004 (DE) 10 2004 008 575.7

(51) Int. Cl. B01J 8/00; C07C 237/16; C07C 55/00; C07C 69/74

(52) U.S. Cl. 422/187; 560/1; 562/590; 564/204

(57) ABSTRACT

The present invention relates to a process for cleaving a (meth)acrylic acid oligomer of structure I 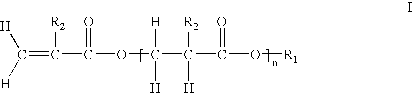 wherein
R₁ is a hydrogen atom or a C₁ to C₁₀ alkyl groups,
R₂ is a hydrogen atom or a methyl group, and n is a whole number within the range between 1 and 200,
   optionally in the presence of a cleaving agent of the structure R₃—OH or of the structure (R₄)₂—N—H, whereby R₃ is a hydrogen atom, a C₁ to C₁₂ alkyl group, or a —CₓH₂ₓ—OH— group, whereby x is a whole number within a range from 1 to 12, and R₄ is a hydrogen atom or a C₁ to C₁₂ alkyl group, with the proviso that not both R₄ groups are hydrogen atoms, whereby the (meth)acrylic acid oligomers are heated with the cleaving agent to a temperature of at least 50° C. at a pressure of at least 1 bar. The present invention also relates to the use of water, optionally with a protic compound as cleaving agent for cleaving (meth)acrylic acid oligomers, a device for (meth)acrylic acid synthesis, the use of this device for production of (meth)acrylic acid, as well as (meth)acrylic acid which has been produced using this device.

This application is a national stage application under 35 U.S.C. 371 of international application No. PCT/EP2005/001705 filed Feb. 18, 2005, published Sep. 1, 2005 as WO 2005/080308 A1, and claiming priority to German Application No. DE 10 2004 008 575.7 filed Feb. 19, 2004, the disclosures of which are expressly incorporated herein by reference.

The present invention relates to a process for cleaving (meth)acrylic acid oligomers in liquid phase, the use of water, optionally with a protic compound, as a cleaving agent for cleaving (meth)acrylic acid oligomers, a device for (meth)acrylic acid synthesis, the use of this device for the production of (meth)acrylic acid as well as (meth)acrylic acid which has been produced using this device.

“(Meth)acrylic acid” is used in this text for the compounds with the nomenclature names “methacrylic acid” and “acrylic acid.” Of the two compounds, one aspect according to the present invention relates to acrylic acid. Furthermore, in this text, the term “oligomer(s)” is used for compounds that comprise two or more repeats of an atom sequence in one molecule. In particular, molecules that are based on at least two monomers, in particular (meth)acrylic acid, are covered by this term.

Acrylic acid is commonly obtained by catalytic gas phase oxidation of propylene with an oxygen-containing gas. Thus, in a two-step process, the propylene is oxidized catalytically in a first process step to acrolein, which is then converted to acrylic acid in a second process step, likewise using catalysts. The thus-obtained acrylic acid is removed from the gaseous reaction mixture in the form of an aqueous solution by absorption with water. The purification of the acrylic acid then occurs by any one of distillation of the acrylic acid solution in a rectification column, extraction with suitable extraction agents, or crystallization processes. In a similar manner, the synthesis of methacrylic acid occurs by catalytic oxidation of any one of isobutylene, t-butanol, methacrolein, or isobutyraldehyde in the gas phase.

(Meth)acrylic acid, however, very quickly tends to form oligomers or even to polymerize, so that, above all in the oxidation of the above-mentioned starting compounds, but also in the distillative work-up of the (meth)acrylic acid solution, (meth)acrylic acid oligomers, such as, for example, (meth)acrylic acid dimers or (meth)acrylic trimers often form as undesired side products. Through the formation of these compounds, the yield of monomeric (meth)acrylic acid in the production of (meth)acrylic acid is observably reduced. In addition to the (meth)acrylic acid synthesis, the formation of (meth)acrylic acid oligomers, which in this case are present in the form of esters, also occurs in a production of (meth)acrylic acid esters by conversion of (meth)acrylic acid with suitable alcohols by heating in the presence of catalysts.

The presence of (meth)acrylic acid oligomers is disadvantageous, in particular, in the production of absorbent polymers and the absorbent polymers obtainable thereby. Thus, with increasing content in (meth)acrylic acid oligomers, the amount of residual monomers present in the absorbent polymer after its production increases. This is disadvantageous, in particular, in the use of the absorbent polymers in the area of baby hygiene articles, since, with diapers, particularly high demands are made on the purity of the polymers used in the diapers.

Discarding the (meth)acrylic acid oligomers is, however, uneconomical. In particular, it is linked with a large loss of (meth)acrylic acid. Numerous processes are, therefore, described in the state of the art that should enable a cleaving of the (meth)acrylic acid oligomers into (meth)acrylic acid monomers and thus the recovery of the (meth)acrylic acid. Continuous and non-continuous processes are used in which the (meth)acrylic acid oligomers in the gas phase or in the liquid phase are cleaved in the presence or in the absence of catalysts, generally at increased temperatures and under pressure.

U.S. Pat. No. 4,317,926 describes the non-catalytic cleaving of acrylic acid dimers in liquid phase at a pressure of 20 to 500 mmHg and at a temperature within a range from 120 to 220° C. Residence times of the dimers in the cleaving reactor within a range from 3 to 8 hours are necessary. Inorganic copper compounds favor the cleaving in the process described in this document.

U.S. Pat. No. 5,734,075 describes the non-catalytic cleaving of acrylic acid dimers in the gas phase at a temperature within a range from 140 to 260° C. The rate of monomer recovery improves in the process described in this document if mixtures of residues from the acrylic acid synthesis and the acrylic acid ester synthesis are used. The residence time of the dimers in the cleaving reactor lies between 0.5 and 3 hours, whereby up to 80 wt. % of the dimers are cleaved. This document does not disclose that the cleaving is carried out at increased pressure.

U.S. Pat. No. 3,086,046 describes the non-catalytic, continuous cleaving of acrylic acid at a pressure of 5 to 150 mmHg and at a temperature within a range from 350 to 650 C. The residence time of the dimers in the cleaving pipe lies within a range between 0.5 and 2 s. The process described in this document is, however, only suitable for acrylic acid residues that have a low molecular weight (at most acrylic acid dimers).

U.S. Pat. No. 3,868,410 describes the cleaving of oligomers that are formed during the esterification of acrylic acid monomers with an alcohol. The cleaving occurs by conversion of the bottom product formed in the esterification reaction with suitable acid catalysts. The use of water in the cleaving reaction is not disclosed.

EP-A-0 751 759 describes the catalytic cleaving of acrylic acid dimers in the gas phase by means of a cycle reactor with a fixed bed at a pressure within a range from 100 to 250 mbar and at a temperature within a range from 200 to 400° C. As catalysts, oxides of the alkali or alkali earth metals, such as, for example, MgO, are used.

An aspect of an embodiment of the present invention relates to providing an improved process for cleaving (meth)acrylic acid oligomers compared to the processes described in the art.

An aspect of another embodiment of the present invention relates to providing a process in which effective oligomer cleaving can occur, also in another aspect without a use of metallic catalysts.

An aspect of a further embodiment of the present invention relates to providing a process with which (meth)acrylic acid esters or (meth)acrylic acid amides can be obtained selectively from (meth)acrylic acid oligomers.

A further aspect of an embodiment of the present invention relates to providing a process for cleaving (meth)acrylic acid oligomers that not only facilitates the cleaving of the oligomers into (meth)acrylic acid but also enables a cleaving of the oligomers to form monomeric (meth)acrylic acid esters.

The various aspects of the various embodiments of the present invention include a process, a device, a (meth)acrylic acid, by fibers, formed articles, films, foams, superabsorbent polymers or hygiene articles and the following details. Further aspects of the various embodiments of the present invention are the subject of the following details, which can be applied individually or combined with each other in any way.

These various aspects of the various embodiments of the present invention include a process for cleaving (meth)acrylic acid oligomers of structure I 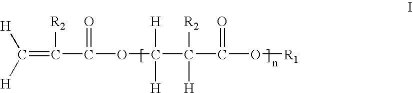
wherein
R₁ is a hydrogen atom or in one aspect a C₁ to C₁₀ alkyl group, in another aspect a C₂ to C₈ alkyl group, and in a further aspect a C₂ to C₄ alkyl group,
R₂ is a hydrogen atom or a methyl group, and
n in one aspect is a whole number within a range between about 1 and about 20, in another aspect within a range from about 1 to about 15, and in a further aspect within a range from about 1 to about 10,
   wherein the (meth)acrylic acid oligomers are heated in one aspect to a temperature of at least about 50° C., in another aspect to a temperature of at least about 150° C., and in yet a further aspect to a temperature of at least about 250° C., whereby in one aspect a temperature of about 500° C. is not exceeded, in another aspect a temperature of about 400° C. is not exceeded, and in yet a further aspect a temperature of about 300° C. is not exceeded, in one aspect at a pressure of at least about 1 bar, in another aspect a pressure of at least about 10 bar, and in yet a further aspect a pressure of at least about 80 bar, whereby in one aspect a pressure of about 1,000 bar is not exceeded, in another aspect a pressure of about 800 bar is not exceeded, and in yet another aspect a pressure of about 600 bar is not exceeded.

In an aspect of an embodiment of the process according to the present invention, the cleaving of (meth)acrylic acid oligomers occurs in the presence of a cleaving agent. Aspects of embodiments of the present invention accordingly also relates to a process for cleaving a (meth)acrylic acid oligomers of structure I 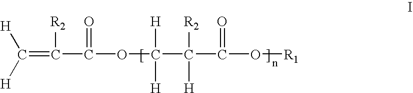
wherein
R₁ is a hydrogen atom or in one aspect a C₁ to C₁₀ alkyl group, in another aspect a C₂ to C₈ alkyl group, in yet a further aspect a C₂ to C₄ alkyl group,
R₂ is a hydrogen atom or a methyl group, and
n in one aspect is a whole number within a range between about 1 and about 20, in another aspect within a range from about 1 to about 15, and in yet a further aspect within a range from about 1 to about 10,
   with a cleaving agent of structure II
   R₃—O—H
   or of structure III
   (R₄)₂—N—H
   wherein

R₃ is a hydrogen atom, or in one aspect a C₁ to C₁₂ alkyl group, in another aspect a C₂ to C₈ alkyl group, and in yet a further aspect a C₂ to C₄ alkyl group, or a —CₓH₂ₓ-OH group, whereby x is a whole number in one aspect within a range from about 1 to about 12, in another aspect within a range from about 2 to about 8, and in yet a further aspect within a range from about 2 to about 4, and

R₄ is a hydrogen atom or in one aspect a C₁ to C₁₂ alkyl group, in another aspect a C₂ to C₈ alkyl group, and in yet a further aspect a C₂ to C₄ alkyl group, with the proviso that not both residues R₄ are hydrogen atoms,

wherein the (meth)acrylic acid oligomers, in liquid phase, are brought into contact with the cleaving agent in one aspect at a temperature of at least about 50° C., in another aspect at a temperature of at least about 150° C., and in yet a further aspect at a temperature of at least about 250° C., whereby in one aspect a temperature of about 500° C. is not exceeded, in another aspect a temperature of about 400° C. is not exceeded, and in yet a further aspect a temperature of about 300° C. is not exceeded, and at a pressure in one aspect of at least about 1 bar, in another aspect of at least about 10 bar, and in yet a further aspect of at least about 80 bar, whereby in one aspect a pressure of about 1,000 bar is not exceeded, in another aspect a pressure of about 800 bar is not exceeded, and in yet a further aspect a pressure of about 600 bar is not exceeded.

In one aspect of an embodiment of the process according to the present invention, a compound of structure II is used as a cleaving agent, whereby the cleaving agent of structure II can be a mixture of at least two structurally different compounds of structure II, whereby this mixture is based on water (R₃═H) to in one aspect at least about 10 wt. %, in another aspect at least about 50 wt. %, and in yet a further aspect at least about 80 wt. %, and in yet another aspect at least about 95 wt. %, respectively, based on the cleaving agent. In another aspect of an embodiment of the process according to the present invention, pure water is used as a cleaving agent. The cleaving agents of structure II can be, in addition to water (R₃═H), an alcohol such as, for example, methanol, ethanol, 1-propanol, 2-propanol, t-butanol, n-butanol, iso-butanol or sec-butanol or a diols such as, for example, ethylene glycol, propylene glycol, or butylene glycol. In a further aspect, cleaving agents of structure II can be mixtures of at least two of the above-mentioned cleaving agents, such as, for example, mixtures of water and ethanol or mixtures of water and butanol.

In addition to pure water or mixtures of at least two structurally different compounds of structure II, mixtures of the above-defined cleaving agents of structure II with other protic compounds, in particular with cleaving agents of structure III or also with polyols can also be used.

In one aspect of the process according to an embodiment of the present invention, the pressure and temperature conditions during the cleaving reaction can be selected such that all reactants participating in the cleaving reaction are present at least partially in liquid form.

Surprisingly, but nonetheless advantageously, it is possible by means of the above-described process, to cleave (meth)acrylic acid oligomers or their esters, optionally using water or other cleaving agents of structure II or of structure III, at increased temperatures and at increased pressure, to form (meth)acrylic acid (with water as a cleaving agent) or (meth)acrylic acid esters (with alcohols as a cleaving agent) or (meth)acrylic acid amides (with primary or secondary amines as a cleaving agent).

In one aspect of an embodiment, a (meth)acrylic acid dimer (n=1, R₂═H, or CH₃), a (meth)acrylic acid trimer (n=2, R₂═H, or CH₃), or a mixture of these two compounds can be used as (meth)acrylic acid oligomer, whereby the residue R₁ can be selected from hydrogen or the alcohol groups methyl, ethyl, n-propyl, isopropyl, n-butyl, sec-butyl, t-butyl, or isobutyl. A (meth)acrylic acid dimer (n=1, R₂═H or CH₃), a (meth)acrylic acid trimer (n=2, R₂═H or CH₃), or mixtures thereof can be used as (meth)acrylic acid oligomer, whereby the residue R₁ is a hydrogen atom.

In a further aspect of an embodiment of the process according to the present invention, whereby the cleaving occurs in the presence of a cleaving agent, the bringing into contact of the cleaving agent with the (meth)acrylic acid oligomer can occur in the presence of a protic compound of structure II or of structure III, which is different from water. This protic compound, according to an aspect of an embodiment of the process of the present invention, can be used instead of water, and according to another aspect of an embodiment of the process of the present invention, can be used in addition to water.

When a compound of structure II or structure III is used to facilitate a cleaving of an (meth)acrylic acid oligomers of structure I, in addition to the (meth)acrylic acid (R₄═H), which forms when water is used as the cleaving agent, corresponding monomeric (meth)acrylic acid esters (R₅=organic residue with 1 to 12 carbon atoms) or (meth)acrylic acid amides (R₅=organic residue with 1 to 12 carbon atoms) also can be obtained selectively. If esterified oligomers are used as (meth)acrylic acid oligomers (R₁=alkyl group or alcohol group), a transesterification of the correspondingly formed monomers of the (meth)acrylic acid oligomers to form the desired monomeric (meth)acrylic acid esters can be carried out selectively by using the compounds of structure II.

If a cleaving agent is used, the cleaving agent and the (meth)acrylic acid oligomer can be used in a weight ratio cleaving agent:(meth)acrylic acid oligomer in one aspect within a range from about 0.01:1 to about 10:1, in another aspect within a range from about 0.1:1 to about 8:1, and in yet a further aspect within a range from about 0.5:1 to about 6:1.

In another aspect of an embodiment of the process according to the present invention, in which the cleaving occurs after the addition of a cleaving agent, the cleaving agent is used in a molar amount that is in one aspect at most about 90%, in another aspect at most about 80%, and in yet a further aspect at most 50% of the molar amount of (meth)acrylic acid that is bound in oligomeric form in the (meth)acrylic acid oligomers (two (meth)acrylic acid molecules in a dimer, three (meth)acrylic acid molecules in a trimer, etc.).

In a further aspect of an embodiment of the process according to the present invention, the cleaving agent is used in a molar amount that is in one aspect at least about 50%, in another aspect at least about 80%, and in yet a further aspect at least about 90% of the molar amount of the (meth)acrylic acid that is bound in oligomeric form in the (meth)acrylic acid oligomers.

In general, the skilled person will determine the amount of cleaving agent desired for the cleaving by suitable trials, if the addition of a cleaving agent for cleaving the (meth)acrylic acid oligomers is necessary. If, for example, pure water is used as cleaving agent, in order to convert the (meth)acrylic acid oligomers into (meth)acrylic acid monomers, the skilled person will continue to add water until, at the selected pressure and temperature conditions, as complete a cleaving as possible has occurred or until no more formation of monomeric (meth)acrylic acid can be observed on further addition of water. If alcohols of structure II are used as a cleaving agent, in order to convert the (meth)acrylic acid oligomers into the corresponding (meth)acrylic acid esters, the skilled person will likewise add these alcohols until as complete as possible a cleaving of the oligomers has occurred or until no further formation of monomeric (meth)acrylic acid or monomeric (meth)acrylic acid esters occurs even upon further addition of alcohol.

By means of the cleaving of the (meth)acrylic acid oligomer using compounds of structure II or of structure III, monomeric compounds of structure IV 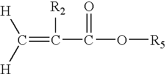
or of structure V 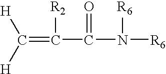
are separated,
whereby
R₆ is a hydrogen atom or in one aspect a C₁ to C₁₂ alkyl group, in another aspect a C₂ to C₈ alkyl group, and in yet a further aspect a C₂ to C₄ alkyl group, with the proviso that not both R₆ groups are hydrogen atoms,
R₅ is a hydrogen atom, or in one aspect a C₁ to C₁₂ alkyl group, in another aspect a C₂ to C₈ alkyl group, and in yet a further aspect a C₂ to C₄ alkyl group, or a —CₓH₂ₓ-OH— group, whereby x is a whole number in one aspect within a range from about 1 to about 12, in another aspect within a range from about 2 to about 8, and in yet a further aspect within a range from about 2 to about 4;
R₂ is a hydrogen atom or a methyl group.

In a further aspect of an embodiment of a process according to the present invention, the (meth)acrylic acid oligomer is used in the form of a composition which is obtained as a bottom product of the distillative work-up of the (meth)acrylic acid solution in process step iii) during the process for (meth)acrylic acid synthesis comprising the process steps:

i) catalytic oxidation of C₃ or C₄ starting compounds in the gas phase;

ii) absorption or condensation or both of the formed (meth)acrylic acid in water; and

iii) work-up of the thus-obtained aqueous (meth)acrylic acid solution by distillation.

Furthermore, in the above process for (meth)acrylic acid synthesis, instead of process step iii), a crystallization step iv) can be provided. In this crystallization step iv), according to an aspect of an embodiment of the present invention, the aqueous (meth)acrylic acid solution can be freed from impurities such as (meth)acrylic acid oligomers. In this crystallization step iv), according to another aspect of an embodiment of the present invention, the (meth)acrylic acid purified by the distillation can be further purified in that impurities such as (meth)acrylic acid oligomers are separated. Common to both aspects of an embodiment is that the impurities such as (meth)acrylic acid oligomers accumulate in the mother liquors and flow-off of these crystallization steps and can be communicated to the process according to aspects of an embodiment of the present invention for cleaving (meth)acrylic acid oligomers.

Furthermore, the composition that accumulates as waste in the bottoms at the different positions of the (meth)acrylic acid synthesis during purification and separation steps can be supplied to the process according to aspects of an embodiment of the present invention for cleaving (meth)acrylic acid oligomers.

According to aspects of an embodiment of the present invention, this composition and/or this bottom product comprise:
(α1) in one aspect about 0.1 to about 70 wt. %, in another aspect about 5 to about 60 wt. %, and in yet a further aspect about 10 to about 50 wt. % monomeric (meth)acrylic acid, as α1-compound;
(α2) in one aspect about 1 about 90 wt. %, in another aspect about 10 to about 40 wt. %, and in yet a further aspect about 20 to about 30 wt. % (meth) acrylic acid dimers, as α2-compound;
(α3) in one aspect about 1 to about 25 wt. %, in another aspect about 2 to about 20 wt. %, and in yet a further aspect about 5 to about 15 wt. % (meth)acrylic acid trimers, as α3-compound;
(α4) in one aspect 0 to about 20 wt. %, in another aspect about 1 to about 10 wt. %, and in yet a further aspect about 2 to about 8 wt. % water, as α4-compound;
(α5) in one aspect about 1 to about 92 wt. %, in another aspect about 10 to about 75 wt. %, and in yet a further aspect about 40 to about 57 wt. % oligomers which are larger than (meth)acrylic acid trimers, as α5-compound; as well as
(α6) in one aspect about 1 to about 20 wt %, in another aspect about 2 to about 15 wt. %, and in yet another aspect about 5 to about 10 wt. % further compounds which are different from the α1-compounds, α2-compounds, α3-compounds, α4-compounds, and α5-compounds, as side products (α6),
   whereby the sum of the components (α1) to (α6) is about 100 wt. %.

The side products (α6) can be those side products that are formed during the catalyzed oxidation of propylene with oxygen in the gas phase in addition to the main product acrylic acid or during the oxidation of C₄ starting compounds, such as, for example, isobutene, isobutane, t-butanol or methacrolein, in addition to the methacrylic acid. In the case of the production of acrylic acid from propylene, these side products (α6) include low boiling, organic compounds, whose boiling point lies below the boiling point of acrylic acid, such as, for example, acrolein, acetic acid or formaldehyde as well as high boiling organic compounds, whose boiling point lies above the boiling point of acrylic acid, such as, for example, maleic acid, maleic acid anhydride, furfuryl aldehyde or benzaldehyde. In the case of the production of methacrylic acid, among these side products (α6) are acetic acid, propionic acid, aldehydes, and maleic acid anhydride.

If the (meth)acrylic acid oligomers are used in the form of the above-described composition, both a continuous and a non-continuous procedure is possible. Thus, the bottom product accumulating in the distillative work-up of the aqueous (meth)acrylic acid solution is continuously removed and, if the cleaving requires the addition of a cleaving agent, transferred using a pump into a mixing device. The continuous removal of the bottom fluid in the present invention means that the removal can occur in portions in constant or not constant time intervals, as well as continuously with constant speed.

The cleaving agent also may be introduced into the mixing device, such as by means of a pump. If a cleaving agent mixture comprising at least two structurally different cleaving agents is used, the individual cleaving agents may be mixed separately from each other with the composition comprising the (meth)acrylic acid oligomer or may be used together as a mixture in the process according to the present invention.

After the components have been mixed in the mixing device, they are heated to a temperature of in one aspect at least about 50° C., in another aspect at least about 150° C., and in yet another aspect at least about 250° C., whereby in one aspect a temperature of about 500° C. is not exceeded, in another aspect a temperature of about 400° C. is not exceeded, and in yet another aspect a temperature of about 300° C. is not exceeded. The heating occurs in one aspect at a pressure of at least about 1 bar, in another aspect of at least about 10 bar, and in yet another aspect of at least about 80 bar, whereby in one aspect a pressure of about 1,000 bar is not exceeded, in another aspect a pressure of about 800 bar is not exceeded, and in yet another aspect a pressure of about 600 bar is not exceeded. The combined components can be heated using a heat exchanger. It is also conceivable first to heat the individual components at the above-mentioned pressures and then to combine them.

The combined and heated components are then cleaved in a cleaving device. This cleaving device can be spatially separated from the mixing device. It is also conceivable that the combining of the components and the subsequent cleaving of the (meth)acrylic acid oligomers occurs in the same device unit.

If no cleaving agent is added, the (meth)acrylic acid oligomers are heated in the cleaving device to the above-mentioned temperatures at the above-mentioned pressures without prior mixing with a cleaving agent and cleaved in this way.

The cleaving of the (meth)acrylic acid oligomers can occur at the above-mentioned temperatures and pressures. In this way, economically advantageous yields can be obtained.

The residence time of the (meth)acrylic acid oligomers in the cleaving reactor can lie in one aspect within a range from about 0.1 seconds to about 20 minutes, in another aspect within a range from about 1 second to about 15 minutes, and in yet another aspect within a range from about 1 to 10 about minutes. In one aspect at least about 30 wt. %, in another aspect at least about 60 wt. %, and in yet a further aspect at least about 70 wt. %, as well as in even yet another aspect at least about 90 wt. % of the (meth)acrylic acid oligomers used can be present after leaving the cleaving reactor as compounds of the structure IV or V.

It is known from the prior art that the crude (meth)acrylic acid obtained in the distillative work-up, which is obtained by absorption of the acrylic acid from the gaseous reaction mixture using water, can be further purified in crystallization processes. The mother liquor obtainable after crystallization of (meth)acrylic acid can still comprise considerable amounts of (meth)acrylic acid oligomers that can likewise be cleaved using the above-described process.

In an aspect of another embodiment of the process according to the present invention, the (meth)acrylic acid oligomer is used in the form of a composition that is obtained as mother liquor during the purification by crystallization in process step IV) during the process for (meth)acrylic acid synthesis comprising the process steps:
I) catalytic oxidation of C₃ or C₄ starting compounds in the gas phase;
II) absorption or condensation or both of the formed (meth)acrylic acid in water to form an absorption product;
III) optionally, work-up of the thus-obtained aqueous (meth)acrylic acid solution by distillation; and
IV) purification by crystallization of the absorption product or of the concentrated (meth)acrylic acid solution obtained by distillation or of both.

In an aspect of an embodiment of the process according to the present invention, this also comprises step III) as a step.

This mother liquor can comprise at most about 65 wt. % (meth)acrylic acid. The portion of (meth)acrylic acid oligomers in the mother liquor lies in one aspect within a range from about 0.1 to about 50 wt. %, in another aspect within a range from about 0.5 to about 50 wt. %, and in yet another aspect within a range from about 1 to about 30 wt. %, respectively based on the total weight of the composition.

A continuous as well as a discontinuous procedure is also possible in this case. Thus, the mother liquor accumulating during the crystallization of the (meth)acrylic acid solution is continuously removed and, if a cleaving agent is added, it is transferred using a pump into a mixing device. The mother liquor, in the case of the continuous carrying out of the process according to the invention, can be removed both in portions in constant or non-constant time intervals as well as continuously with constant speed. The further steps of this process correspond to those process steps or aspects respectively that have already been described in the context of the use of the bottom product of the distillative work-up of the aqueous acrylic acid solution as starting material for a process according to various aspects of embodiments of the present invention for cleaving (meth)acrylic acid oligomers.

The cleaving of the (meth)acrylic acid oligomers can occur in the presence of a catalyst. Catalysts can be metal catalysts, such as, for example, catalysts based on antimony, cobalt, or manganese; acid salts; inorganic acid catalysts, such as, for example, sulphuric acid or hydrochloric acid; organic acid catalysts, such as, for example, p-tolunesulfonic acid or methanesulfonic acid or both; hydroxides, such as, for example, potassium hydroxide, lithium hydroxide, antimony hydroxides, cobalt hydroxides, manganese hydroxides or lead hydroxides; metal salts, such as, for example, zinc chlorides; or mixtures of at least two thereof. The catalyst can be used in pure form or immobilized on a substrate, for example, in combination with zeolites, which can be water-resistant, or ion exchange resins. The catalyst can be used in one aspect in an amount within a range from about 1 to about 5,000 ppm, in another aspect in an amount within a range from about 10 to about 2,000 ppm, and in yet another aspect in an amount within a range from about 100 to about 1,000 ppm, based on the (meth)acrylic acid oligomers.

Also, aspects of embodiments of the present invention relate to the use of compounds of structure II or structure III, whereby R₃ and R₄ are defined as above, as cleaving agent for cleaving (meth)acrylic acid oligomers of structure I in one aspect at a temperature of at least about 50° C., in another aspect at a temperature of at least about 150° C., and in yet a further aspect at a temperature of at least about 250° C., whereby in one aspect a temperature of about 500° C. is not exceeded, in another aspect a temperature of about 400° C. is not exceeded, and in yet a further aspect a temperature of about 300° C. is not exceeded, and in one aspect at a pressure of at least about 1 bar, in another aspect of at least about 10 bar, and in yet a further aspect of at least about 100 bar, whereby in one aspect a pressure of about 1,000 bar is not exceeded in liquid phase, in another aspect a pressure of about 800 bar is not exceeded in liquid phase, and in yet a further aspect a pressure of about 600 bar is not exceeded in liquid phase. In particular, aspects of embodiments of the present invention relate to the use of water, alcohols such as ethanol or butanol or mixtures of water and ethanol or water and butanol as a cleaving agent for cleaving compounds of structure I under the above-mentioned pressure and temperature conditions.

Aspects of embodiments of the present invention further relate to a device for production of (meth)acrylic acid comprising as components connected with each other in fluid-communicating fashion a (meth)acrylic acid synthesis unit, a quench absorber or condensation device, a distillation device and/or a crystallization device as well as a (meth)acrylic acid oligomer cleaving device, whereby the (meth)acrylic acid oligomer cleaving device comprises a cleaving agent reservoir, a first and a second conveyor unit, a mixing device, a heating device, a cleaving reactor, which can be made from high-alloy steels, in particular steels based on nickel, and at least a first to fifth conduit, whereby:
(β1) the first conveyor unit comprises a feed line, which communicates a composition as defined above comprising a (meth)acrylic acid oligomer;
(β2) the cleaving agent reservoir communicates via a first conduit with the second conveyor unit;
(β3) the first and the second conveyor unit communicate with the mixing device via a second and third conduit;
(β4) the mixing device communicates with the heating device via a fourth conduit;
(β5) the heating device communicates with the cleaving reactor via a fifth conduit.

According to the present invention, by “fluid-communicating,” it is understood that gases or liquids or mixtures thereof are transportable, as is possible through, for example, lines, and/or pumps or the like.

A cleaving reactor may be any reactor type known to those skilled in the art that can be operated at the above-mentioned pressure and temperature conditions. Heating devices may be pipe reactors, pipe bundle reactors, and/or Taylor reactors.

In one aspect of an embodiment relating to an oligomer cleaving device according to the present invention, at least two selected from a mixing device, a heating device and a cleaving reactor form a spatial unit. In another aspect of an embodiment relating to an oligomer cleaving device according to the present invention, a mixing device, a heating device, and a cleaving reactor form a spatial unit. In this context, a spatial unit means that, for example, a mixing device and a heating device are present together in a section, and the mixing and heating step occurs at the same position. Thus, in yet another aspect of an embodiment relating to an oligomer cleaving device according to the present invention, the heating device and the cleaving reactor are present together in a section.

Further aspects of an embodiment relating to an oligomer cleaving device according to the present invention are that at the cleaving reactor, (β6), a condensation device communicates with the cleaving reactor via a sixth conduit. In one aspect of an embodiment with such a condensation device, the (meth)acrylic acid can be separated from the higher boiling impurities. In another aspect, the condensation device is operated at a lower pressure than the cleaving reactor. In yet another aspect, the crude product forming in the cleaving reactor is expanded in the condensation device that in a further aspect occurs in the presence of a protective gas, such as, nitrogen or argon. It is, however, also conceivable to expand the crude product arising in the cleaving reactor before reaching the condensation device in an expansion device that is separate from the condensation device, and to separate the liquid phase obtained after the expansion from the gaseous phase present in the expansion device, whereby the separation of the liquid phase from the gaseous phase can occur using separating devices known to those skilled in the art, such as, for example, a cyclone. The liquid phase, which still comprises large amounts of (meth)acrylic acid oligomers, can be supplied back to the cleaving device after renewed compression. The gaseous phase, which can still comprise small amounts of (meth)acrylic acid oligomers in addition to monomeric (meth)acrylic acid, can then be supplied to the condensation device or to another purification device, for example to a distillation column, for further purification. In a further aspect, the higher boiling impurities separated in the condensation device can be supplied again to the first conveying unit. This can occur in an aspect with incomplete cleaving of the oligomers. On the other hand, in another aspect with high boilers not comprising monomer, there may be no recycling. In addition, in yet another aspect, the (meth)acrylic acid separated by means of the condensation device, if accompanied by water, can be supplied to a crystallization device for further purification.

A device for production of (meth)acrylic acid in aspects of an embodiment comprises the following construction in the area that comprises a (meth)acrylic acid synthesis unit and a quench absorber for the synthesis of acrylic acid: propylene and, optionally, further inert gases, such as, for example, nitrogen or combustion gases, such as, for example, CO₂ or nitrogen oxides, are communicated via a reactant feed line to a first reactor for a first catalytic oxidation. The first reactor communicates via a further line with a second reactor, into which the product of the first catalytic oxidation from the first reactor is communicated for a second catalytic oxidation. The acrylic acid-comprising product of the second catalytic oxidation is communicated via a feed line located between the second reactor and the quench absorber to the lower half of the quench absorber. In the quench absorber, the product of the second catalytic oxidation is brought into contact with water, whereby the water is introduced into the quench absorber above the feed line of the product of the second catalytic oxidation. On the one hand, a first phase (=aqueous acrylic acid solution) comprising acrylic acid and water is communicated away from the quench absorber below the supply of the product of the second catalytic oxidation. The first phase can be at least partially communicated back into the quench absorber. The first phase not communicated back into the quench absorber is supplied to the distillation device, for example, in order to be subjected to an azeotropic separation, in which the acrylic acid is concentrated and purified. It is also conceivable that the first phase that is not communicated back into the quench absorber is supplied to the crystallization device, in which a purification of the acrylic acid likewise can occur. It is further possible that the first phase which is not communicated back into the quench absorber is first supplied to a distillation device, and the acrylic acid that has been purified and concentrated by means of the distillation device is then fed to the crystallization device. Above the communicating back of the first phase and below the communicating of water into the quench absorber, a second phase comprising acrylic acid and water can be communicated away from the quench absorber. The second phase, like the first phase, can be communicated to the distillation device or to the crystallization device. The waste gases derived from the quench absorber can be communicated to a catalytic combustor. The combustion gases of the catalytic combustor can be communicated as inert gases into the first reactor. The water recovered from the concentration of acrylic acid can be communicated back into the quench absorber. Further details for the production of acrylic acid are disclosed in DE 197 40 252 A1.

A device for the production of methacrylic acid in aspects of an embodiment comprises a (meth)acrylic acid synthesis unit and the quench absorber for the synthesis of methacrylic acid by catalytic gas phase oxidation of C₄ starting compounds with oxygen. In one aspect, methacrylic acid is obtainable by catalytic gas phase oxidation of isobutene, isobutane, t-butanol, isobutyraldehyde, methacrolein, or methyl-t-butyl ether. Further details are disclosed in EP 0 092 097 B1, EP 0 058 927, and EP 0 608 838.

In one aspect of an embodiment of a device according to the present invention, the composition that is communicated in the feed line to the first conveyor unit corresponds to that composition which is obtained as bottom product from the distillative work-up of the (meth)acrylic acid solution.

In another aspect of an embodiment of a device according to the present invention, the composition that is communicated in the feed line to the first conveyor unit corresponds to that composition which is obtained as mother liquor in the purification of the distillate by crystallization.

A further another aspect of an embodiment of the present invention relates to the use of the above-described device for the production of (meth)acrylic acid.

The invention also relates to the use of (meth)acrylic acid, obtainable by the use of the above-described device, in the production of fibers, formed articles, films, foams, leather additives and paper additives, and detergents, as well as superabsorbent polymers or hygiene articles.

Aspects of embodiments of the present invention are more closely illustrated in the nonlimiting figures described below:

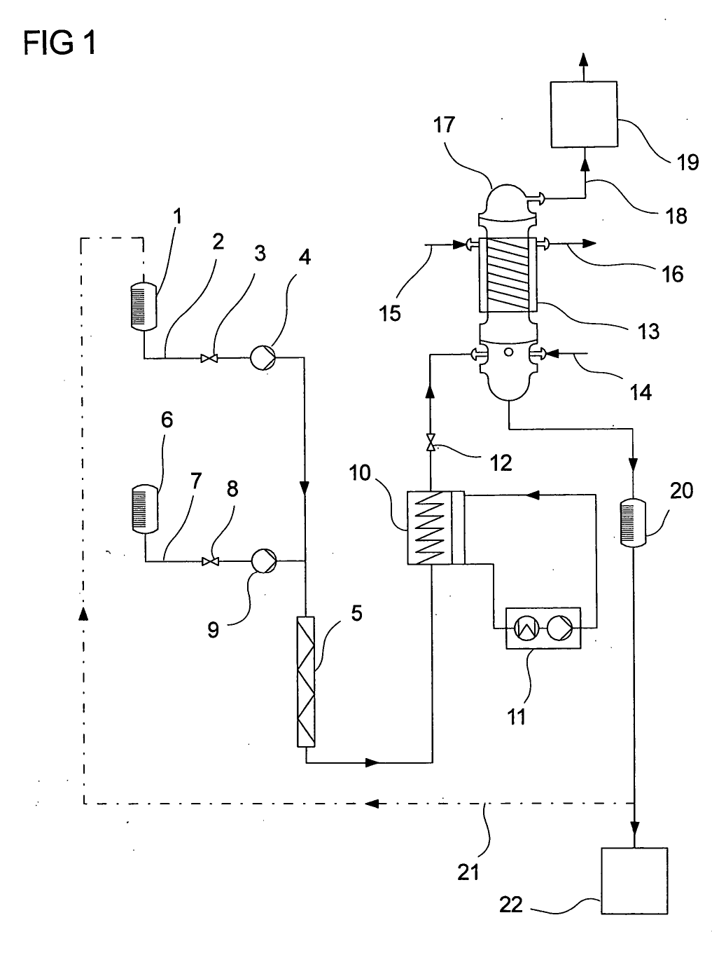

FIG. 1 shows a schematic representation of a device including an oligomer cleaving device according to an aspect of an embodiment of the present invention;

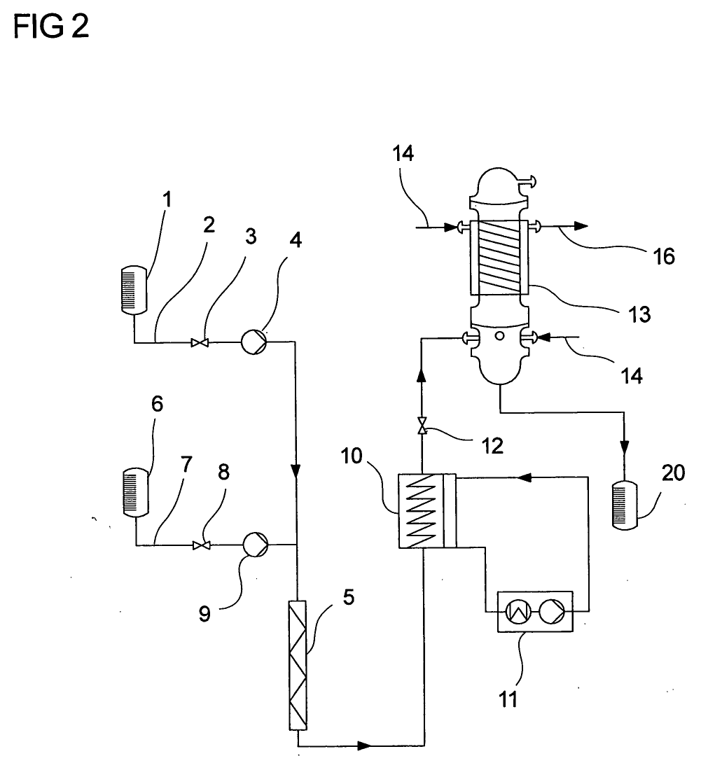

FIG. 2 shows a schematic representation of a device including an oligomer cleaving device used in the examples according to an aspect of an embodiment of the present invention; and

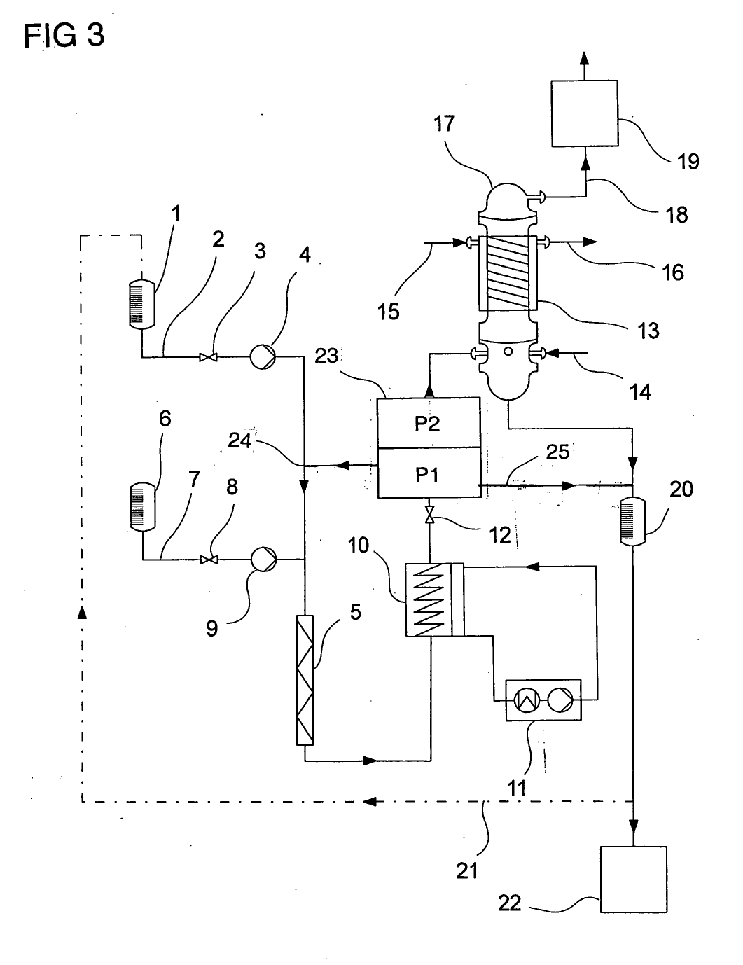

FIG. 3 shows a schematic representation of a particular design of a device including the oligomer cleaving device according to an aspect of an embodiment of the present invention.

A composition comprising (meth)acrylic acid oligomers held in a reactant tank 1, shown in FIG. 1, is supplied via a reactant line 2, which is regulated by a reactant valve 3, to a reactant pressure pump 4 acting as a first conveyor unit. By means of a reactant pressure pump 4, the composition comprising (meth)acrylic acid oligomers is compressed and supplied to a mixing device 5. If no additional cleaving agent is used, the mixing device can be dispensed with. The cleaving agent situated in a cleaving agent reservoir 6 is fed via a cleaving agent line 7, which is regulated by a cleaving agent valve 8. to a cleaving agent pressure pump 9 acting as a second conveyor unit. The cleaving agent pressure pump 9 compresses the cleaving agent and communicates it to the mixing device 5. The mixture of reactant and cleaving agent in the mixing device 5 is communicated to a heating device 10 comprising a cleaving reactor. The heating device 10 is heated by means of a heat exchanger 11. The cleaving reaction product of the (meth)acrylic acid oligomer cleaving situated in the heating device 10 is released from pressure by means of a pressure release valve 12 and communicated to a condenser 13. Protecting gas is communicated to the condenser 13 by means of a protecting gas feed 14. The condenser 13 is cooled by means of a cooling agent supply 15 and a cooling agent discharge 16, so that high boilers are concentrated in the lower region of the condenser 13, and in a condenser head 17, (meth)acrylic acid is optionally enriched with water that is supplied by means of a pure product line 18 to a crystallization device 19 in which the (meth)acrylic acid is separated from the associated water and further purified. The crystallization device 19 can also be a distillation or condensation device. In the lower region of the condenser 13, high boilers are transferred into a high boiler tank 20 and, on the one hand, can be communicated back into reactants tank 1 or, on the other hand, be supplied to a high boiler disposal 22.

FIG. 2 represents the experimental construction used in the following examples. Concerning the individual parts of the oligomer cleaving device, reference is made to the details of FIG. 1.

FIG. 3 represents a particular aspect of an embodiment of a cleaving device according to the present invention, in which the release of pressure of the cleaving product is not carried out in condenser 13, as shown in FIG. 1, but in an expansion device 23 (flash device) which is separate from the condenser. After the release of pressure, a liquid phase P1 and a gaseous phase P2 are obtained in the flash device 23 (see FIG. 3). The gaseous phase P2, which can still comprise small amounts of oligomers in addition to monomeric (meth)acrylic acid, can then be introduced into a further purification device 13, which can be an evaporator or a distillation column, whereby optionally a condensation of the components of the gaseous phase P2 can be carried out before the introduction. The liquid phase P1, which still comprises large amounts of (meth)acrylic acid oligomers, can be communicated (e.g., via line 25) to the high boiler disposal 22 or communicated back to the heating device 10 for cleaving. If a cleaving agent is used for cleaving, the communicating back of the liquid phase P1 (e.g., via line 24) to the heating device can occur via a mixing device 5, as is shown in FIG. 3. If no additional cleaving agent is added to the composition to be cleaved, the liquid phase P1 can also be supplied after corresponding compression directly to the heating device 10 (not shown).

The invention is now more closely illustrated by means of non-limiting examples.

### EXAMPLES

A device as shown in FIG. 2 was used, in which before the cleaving reactor two HPLC-pumps were used as conveyor units and a static mixer from the company SULZER with dimensions 80×15.5 mm was used. As a cleaving reactor, a coiled tubing reactor in the Marlotherm bath was used. The pressure of the product under pressure obtained in the cleaving reactor were released via a spring valve from the company Hoke as a pressure release valve and the product was supplied to a condensation in a flash distillation head with an intensive cooler DN50. The product collected in the bottom of the flash distillation head was investigated by means of GC and Karl-Fischer Titration with respect to its components. The degree of cleaving [in %] was determined from the thus-obtained compositions. The degree of cleaving is defined as follows:
Degree of cleaving=100×(number of moles of cleaved dimers/number of moles of the dimers comprised in the composition used)

The (meth)acrylic acid oligomers were used in the form of a composition that was obtained as bottom product in the distillative work-up of an aqueous acrylic acid solution. The compositions are given in the following examples.

Investigation of the influence of temperature on the cleaving

### Examples 1to 3

A bottom product comprising 0.1 wt. % water, 54 wt. % acrylic acid, and 31 wt. % dimeric acrylic acid and obtained in the distillative work-up of an aqueous acrylic acid solution was cleaved at different temperatures in the above-described device. The degree of cleaving was determined.

The following values were determined for the degree of cleaving:

TABLE 1

| | | | Residence | composition [wt. %] of the | | | | Degree of |
|---|---|---|---|---|---|---|---|---|
| Temp. | | Weight ratio | time | composition after the cleaving | | | | cleaving |
| [° C.] | p [bar] | H₂O:Oligomer⁰⁾ | [min] | H₂O | AA¹⁾ | DAA²⁾ | remainder | [%] |
| 180 | 10 | 4.2:1 | 3 | 68 | 21 | 8 | 3 | 25 |
| 240 | 35 | 4.2:1 | 3 | 68 | 25 | 4 | 3 | 67 |
| 280 | 65 | 4.2:1 | 3 | 68 | 25 | 2 | 5 | 82 |

⁰⁾total amount of all oligomers in the composition
¹⁾AA = acrylic acid
²⁾DAA = dimeric acrylic acid

It can be seen from Table 1 that the degree of cleaving increases with increasing temperature.

Investigation of the influence of the amount of water on the cleaving

### Examples 4 to 7

A bottom product comprising 0.1 wt. % water, 54 wt. % acrylic acid, and 31 wt. % dimeric acrylic acid obtained in the distillative work-up of an aqueous acrylic acid solution was cleaved with different amounts of water in the above-described device. The degree of cleaving was determined.

The following values were determined for the degree of cleaving:

TABLE 2

| | | | Residence | Composition [wt. %] of the | | | | Degree of |
|---|---|---|---|---|---|---|---|---|
| Temp. | | Weight ration | time | composition after the cleaving | | | | cleaving |
| [° C.] | p [bar] | H₂O:Oligomer⁰⁾ | [min] | H₂O | AA¹⁾ | DAA²⁾ | remainder | [%] |
| 185 | 12 | 0 | 3 | 1 | 52 | 30 | 17 | −19³⁾ |
| 185 | 12 | 4.2:1 | 3 | 68 | 21 | 8 | 3 | 25 |
| 280 | 65 | 0.48:1 | 3 | 16 | 58 | 15 | 11 | 31 |
| 280 | 65 | 4.2:1 | 3 | 68 | 25 | 2 | 5 | 82 |

³⁾the entry −19 shows that after heating in the cleaving device under pressure, the amount of dimers has increased in the absence of water.

It can be seen from Table 2 that the degree of cleaving increases with increasing amount of water and increasing temperature.

Investigation of the influence of the residence time on the cleaving

### Examples 8 and 9

A bottom product comprising 2 wt. % water, 59 wt. % acrylic acid, and 26 wt. % dimeric acrylic acid and obtained in the distillative work-up of an aqueous acrylic acid solution was cleaved in the above-described device with varying residence time. The degree of cleaving was determined.

The following values were determined for the degree of cleaving:

TABLE 3

| | | | Residence | Composition [wt. %] of the | | | | Degree of |
|---|---|---|---|---|---|---|---|---|
| Temp. | | Weight ratio | time | composition after the cleaving | | | | cleaving |
| [° C.] | p [bar] | H₂O:Oligomer⁰⁾ | [min] | H₂O | AA¹⁾ | DAA²⁾ | remainder | [%] |
| 280 | 65 | 0.48:1 | 3 | 17 | 58 | 15 | 10 | 31 |
| 280 | 65 | 0.45:1 | 11 | 15 | 59 | 15 | 11 | 29 |

It can be seen from Table 3 that the reaction occurs substantially spontaneously and is already finished after about three minutes (at 280° C.).

Determination of the influence of addition of butanol on the cleaving.

### Example 10

A bottom product comprising 0.1 wt. % water, 60 wt. % acrylic acid, and 22 wt. % dimeric acrylic acid and obtained from the distillative work-up of an aqueous acrylic acid solution was cleaved with butanol as a cleaving agent. The degree of cleaving was determined.

The following values were determined for the degree of cleaving:

| | | | Residence | Composition [wt. %] of the | | | | | Degree of |
|---|---|---|---|---|---|---|---|---|---|
| Temp. | | Weight ratio | time | composition after the cleaving | | | | | cleaving |
| [° C.] | P [bar] | Butanol:Oligom.⁰⁾ | [min] | H₂O | AA¹⁾ | BA⁴⁾ | DAA²⁾ | remainder | [%] |
| 280 | 65 | 3.64:1 | 5.6 | 6 | 8 | 31 | 0.5 | 45.5 | 95 |

⁴⁾BA = Butyl acrylate

It can be seen from Table 4 that with the cleaving process according to the invention, a degree of cleaving of 95% can be achieved within about 5 minutes.

1. A process for cleaving (meth)acrylic acid oligomers comprising the steps of:
providing at least one (meth)acrylic acid oligomer comprising a structure I 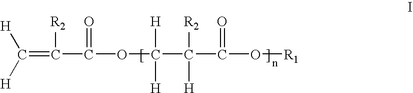
wherein
R₁ is a hydrogen atom or a C₁ to C₁₀ alkyl groups
R₂ is a hydrogen atom or a methyl group, and
n is a whole number within the range between 1 and 200; and
heating the at least one (meth)acrylic acid oligomer to a temperature of at least 50° C. at a pressure of at least 10 bar.

2. A process for cleaving (meth)acrylic acid oligomers comprising the steps of:
providing at least one (meth)acrylic acid oligomer comprising a structure I 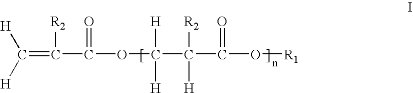
wherein
R₁ is a hydrogen atom or a C₁ to C₁₀ alkyl groups
R₂ is a hydrogen atom or a methyl group, and
n is a whole number within the range between 1 and 200;
providing a cleaving agent comprising a structure II
R₃—OH
or structure III
(R₄)₂—N—H
wherein
R₃ is a hydrogen atom, a C₁ to C₁₂ alkyl group, or a —CₓH₂ₓ-OH group, wherein x is a whole number within a range from 1 to 12, and
R₄ is a hydrogen atom or a C₁ to C₁₂ alkyl group, with the proviso that not both R₄ groups are hydrogen atoms; and
contacting the at least one (meth)acrylic acid oligomer with the cleaving agent at a temperature of at least 50° C. and at a pressure of at least 10 bar.

3. The process according to claim 2, wherein the cleaving agent and the at least one (meth)acrylic acid oligomer are used in a cleaving agent:(meth)acrylic acid oligomer weight ratio within a range from 0.01:1 to 10:1.

4. The process according to claim 2, wherein the cleaving agent comprises any one of water, ethanol, n-butanol, or a mixture of at least two of these compounds.

5. The process according to claim 1, wherein a cleaving product comprises a compound comprising a structure IV 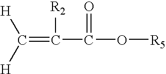
or structure V 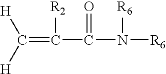
wherein
R₆ is an H atom or a C₁-C₁₂ alkyl group, with the proviso that not both R₆ groups are hydrogen atoms,
R₅ is an H atom, a C₁-C₁₂ alkyl group or a —CₓH₂ₓ-OH— group, whereby x is a whole number within a range from 1 to 12, and
R₂ is an H atom or a methyl group.

6. The process according to claim 1, wherein the least one (meth)acrylic acid oligomer comprises a composition comprising a bottom product of the distillative work-up of a (meth)acrylic acid solution in process step iii) during a process for (meth)acrylic acid synthesis comprising the steps of:
i) catalytically oxidizing C₃ or C₄ starting compounds in a gas phase;
ii) any one of absorbing condensing or absorbing and condensing a formed (meth)acrylic acid in water; and
iii) working-up the thus-obtained aqueous (meth)acrylic acid solution by distillation.

7. The process according to claim 1, wherein the least one (meth)acrylic acid oligomer comprises a composition comprising a mother liquor obtained during the purification by crystallization in process step IV) during a process for (meth)acrylic acid synthesis comprising steps of:
I) catalytically oxidizing C₃ or C₄ starting compounds in a gas phase;
II) any one of absorbing condensing or absorbing and condensing formed (meth)acrylic acid in water to form an absorption product;
III) optionally, working-up the thus-obtained aqueous (meth)acrylic acid solution by distillation; and
IV) purifying by crystallization the absorption product or purifying by crystallization a concentrated (meth)acrylic acid solution obtained by distillation or purifying by crystallization the absorption product and purifying by crystallization a concentrated (meth)acrylic acid solution obtained by distillation.

8. The process according to claim 2, wherein the contacting of the at least one (meth)acrylic acid oligomer with the cleaving agent occurs at a temperature of at least 250° C.

9. The process according to claim 1, further comprising providing a catalyst.

10. Using a compound comprising a structure II
R₃—OH
wherein
R₃ is a hydrogen atom, a C₁ to C₁₂ alkyl group, or a —CₓH₂ₓ—OH group, wherein x is a whole number within a range from 1 to 12, or
structure III
(R₄)₂—N—H
wherein
R₄ is a hydrogen atom or a C₁ to C₁₂ alkyl group, with the proviso that not both R₄ groups are hydrogen atoms,
as a cleaving agent for cleaving at least one (meth)acrylic acid oligomer comprising a structure I 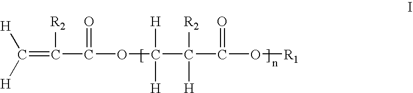
wherein
R₁ is a hydrogen atom or a C₁ to C₁₀ alkyl groups
R₂ is a hydrogen atom or a methyl group, and
n is a whole number within the range between 1 and 200,
at a temperature of at least 50° C. and at a pressure of at least 10 bar.

11. A device for production of (meth)acrylic acid comprising as components connected with each other in fluid-communicating fashion a (meth)acrylic acid synthesis unit, a quench absorber, a distillation device and/or a crystallization device, and a (meth)acrylic acid oligomer cleaving device, wherein the (meth)acrylic acid oligomer cleaving device comprises a cleaving agent reservoir, at least one first and one second conveyor unit, a mixing device, a heating device, a cleaving reactor and at least a first to fifth conduit, wherein
(β1) a reactant pump as the first conveyor unit comprises a feed line, which communicates a composition comprising at least one (meth)acrylic acid oligomer comprising a structure I: 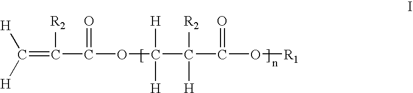
wherein
R₁ is a hydrogen atom or a C₁ to C₁₀ alkyl groups
R₂ is a hydrogen atom or a methyl group, and
n is a whole number within the range between 1 and 200:
(β2) the cleaving agent reservoir communicating by a cleaving agent line as first conduit to a cleaving agent pressure pump as second conveyor unit;
(β3) the first and the second conveyor unit communicating with the mixing device by a second and third conduit;
(β4) the mixing device communicating with the heating device by a fourth conduit; and
(β5) the heating device communicating with the cleaving reactor by a fifth conduit,
wherein the oligomer cleaving device comprises a release valve, by means of which the cleaving product of the (meth)acrylic acid oligomer cleaving situated in the heating device can be expanded.

12. The device according to claim 11, wherein the composition which is communicated in the feed line to the first conveyor unit corresponds to at least one (meth)acrylic acid oligomer comprising a composition comprising a bottom product of the distillative work-up of a (meth)acrylic acid solution in process step iii) during a process for (meth)acrylic acid synthesis comprising the steps of:
i) catalytically oxidizing C₃ or C₄ starting compounds in a gas phase;
ii) any one of absorbing condensing, or absorbing and condensing a formed (meth)acrylic acid in water; and
iii) working-up the thus-obtained aqueous (meth)acrylic acid solution by distillation.

13. The device according to claim 11, wherein the composition which is communicated in the feed line to the first conveyor unit corresponds to at least one (meth)acrylic acid oligomer comprising a composition comprising a mother liquor obtained during the purification by crystallization in process step IV) during a process for (meth)acrylic acid synthesis comprising steps of:
I) catalytically oxidizing C₁ or C₄ starting compounds in a gas phase;
II) any one of absorbing condensing or absorbing and condensing formed (meth)acrylic acid in water to form an absorption product;
III) optionally working-up the thus-obtained aqueous (meth)acrylic acid solution by distillation; and
IV) purifying by crystallization the absorption product or purifying by crystallization a concentrated (meth)acrylic acid solution obtained by distillation or purifying by crystallization the absorption product and purifying by crystallization a concentrated (meth)acrylic acid solution obtained by distillation.

14. Using a device according to claim 11 for a production of (meth)acrylic acid.

15. Using a device according to claim 12 for a production of (meth)acrylic acid.

16. Using a device according to claim 13 for a production of (meth)acrylic acid.

17. The process according to claim 2, wherein a cleaving product comprises a compound comprising a structure IV 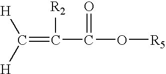
or structure V 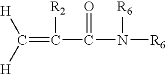
wherein
R₆ is an H atom or a C₁-C₁₂ alkyl group, with the proviso that not both R₆ groups are hydrogen atoms,
R₅ is an H atom, a C₁-C₁₂ alkyl group or a —CₓH₂ₓ-OH— group, whereby x is a whole number within a range from 1 to 12, and
R₂ is an H atom or a methyl group.

18. The process according to claim 2, wherein the at least one (meth)acrylic acid oligomer comprises a composition comprising a bottom product of the distillative work-up of a (meth)acrylic acid solution in process step iii) during a process for (meth)acrylic acid synthesis comprising the steps of:
i) catalytically oxidizing C₃ or C₄ starting compounds in a gas phase;
ii) any one of absorbing, condensing, or absorbing and condensing a formed (meth)acrylic acid in water; and
iii) working-up the thus-obtained aqueous (meth)acrylic acid solution by distillation.

19. The process according to claim 2, wherein the at least one (meth)acrylic acid oligomer comprises a composition comprising a mother liquor obtained during the purification by crystallization in process step IV) during a process for (meth)acrylic acid synthesis comprising steps of:
I) catalytically oxidizing C₃ or C₄ starting compounds in a gas phase;
II) any one of absorbing, condensing, or absorbing and condensing formed (meth)acrylic acid in water to form an absorption product;
III) optionally, working-up the thus-obtained aqueous (meth)acrylic acid solution by distillation; and
IV) purifying by crystallization the absorption product or purifying by crystallization a concentrated (meth)acrylic acid solution obtained by distillation or purifying by crystallization the absorption product and purifying by crystallization a concentrated (meth)acrylic acid solution obtained by distillation.

20. The process according to claim 2, further comprising providing a catalyst.
